(19) United States
(12) Patent Application Publication
(10) Pub. No.: US 2014/0255322 A1
(43) Pub. Date: Sep. 11, 2014

(54) SILOXANE POLYMER AND ACTIVES

(71) Applicants: John D. Blizzard, Bay City, MI (US); Robert L. McKellar, Midland, MI (US)

(72) Inventors: John D. Blizzard, Bay City, MI (US); Robert L. McKellar, Midland, MI (US)

(21) Appl. No.: 14/286,295

(22) Filed: May 23, 2014

Related U.S. Application Data
(63) Continuation-in-part of application No. 13/626,931, filed on Sep. 26, 2012.

(51) Int. Cl. A61K 47/34; A61K 31/165; A61K 31/60; A61Q 9/04; A61K 31/606; A01N 65/44; A61Q 19/00; A61Q 5/02; A61K 8/895; A61K 31/045

(52) U.S. Cl. CPC A61K 47/34; A61K 8/895; A61K 31/165; A61K 31/60; A61K 31/045; A61K 31/606; A01N 65/44; A61Q 19/00; A61Q 5/02; A61Q 9/04
USPC 424/59; 514/772.3; 514/627; 514/159; 514/729; 514/166; 424/750; 424/65; 424/73; 424/70.12; 424/64; 510/122; 8/161

(57) ABSTRACT

A siloxane polymer base that is useful as an excipient for many variable materials that can be compounded into it. Also disclosed are a method for the preparation of such bases, and another method regarding the preparation of the base and actives.

This application is a continuation-in-part of U.S. patent application Ser. No. 13/626,931, filed Sep. 26, 2012, now pending, from which priority is claimed.

### BACKGROUND OF THE INVENTION

The material of this invention is a siloxane polymer that is useful as a base for pharmaceuticals and cosmetics, “Actives” for purposes of this invention means any material that will provide a benefit to a person such as medicaments, such as triple antibiotic, such as pharmaceuticals, such as acetasalicylic acid, and the like and cosmetic materials.

Polysiloxane gels are known from U.S. Pat. No. 5,654,362, that issued on Aug. 5, 1997 to Schulz, et al., which teaches the preparation of siloxane gels using polysiloxane oils.

In that patent, the patentees disclose that the process for producing the polysiloxane gel is carried out by reacting an Si—H containing polysiloxane with an alpha, omega-diene in the presence of platinum catalyst and a low molecular weight silicone oil, wherein a gel is formed.

Thereafter, a paste is formed by subjecting the gel to additional amounts of low molecular weight oil and shear force to form the paste.

Even though the '362 patent, discloses a further step of reducing the paste to a crumble-like product, the instant invention does not deal with that aspect of that disclosure.

The paste is easily formed and handled and can be used in pharmaceutical preparations, cosmetics, and other such materials, but it has a drag and resistance to spreading into a smooth layer when applied to the skin. After application, the paste has a tacky touch which in some cases can be repellent to the user.

Moreover, even though it is disclosed in the '362 patent that the gels, pastes and crumbles can be used as carriers for other materials, such as pharmaceuticals, biocides, herbicides, pesticides, and other biologically active substances there are no examples in the patent to support this allegation.

### THE INVENTION

What is disclosed herein is a method of preparing a siloxane polymer base the method comprising reacting an Si—H containing polysiloxane of the formula selected from the group consisting of
i. R₃SiO(R′₂SiO)ₐ (R″HSiO)_{b} SiR₃
ii. HR₂SiO(R′₂SiO)_{c} SiR₂H, and,
iii. HR₂SiO(R′₂SiO)ₐ (R″HSiO)_{b} SiR₂H, wherein R, r′, R″, are alkyl groups of 1 to 6 carbon atoms; a is 0 to 250; b is 1 to 250; and c is 0 to 250; with an alpha, omega-diene having the general formula CH₂═CH(CH₂)ₓCH═CH₂ wherein x has a value of from 1 to 20.

The reaction is conducted in the presence of a noble metal catalyst and a compound selected from the group consisting of an ester of an alkoxylated aromatic alcohol and a fatty carboxylic acid, or a mixture of such esters, and continuing the reaction until a gel is formed by cross linking and addition of Si—H across double bonds in the alpha, omega-diene.

Thereafter, there is added a first additional component to the gel and the mixture is subjected to shear force until a siloxane paste is formed.

Thereafter, there is added a second additional component to the paste and they are blended by mixing until a siloxane polymer base is formed,

An additional embodiment is the preparation of a siloxane polymer wherein, the process comprises (A), reacting an —Si—H containing polysiloxane of the formula selected from the group consisting of i. R₃SiO(R′₂SiO)ₐ (R″HSiO)_{b} SiR₃, ii. HR₂SiO(R′₂SiO)_{c} SiR₂H, and, iii. HR₂SiO(R′₂SiO)ₐ (R″HSiO)_{b} SiR₂H, wherein R, r′, R″, are alkyl groups of 1 to 6 carbon atoms; a is 0 to 250; b is 1 to 250; and c is 0 to 250; with (B) an alpha, omega-diene having the general formula CH₂═CH(CH₂)ₓ CH═CH₂ wherein x has a value of from 1 to 20; (C) conducting the reaction in the presence of a noble metal catalyst and a compound selected from the group consisting of an ester of an alkoxylated aromatic alcohol and a fatty carboxylic acid, or a mixture of such esters, and continuing the reaction until a gel is formed by cross linking and addition of Si—H across double bonds in the alpha, omega-diene.

In another embodiment, what is disclosed and claimed herein is a composition of matter comprising a siloxane polymer base that is comprised of a siloxane gel, wherein the gel is comprised of a first component selected from esters of alkoxylated aromatic alcohol and fatty carboxylic acids, wherein the second component is a siloxane co-polymer.

The siloxane co-polymer is comprised of the reaction product of a first component hydridopolysiloxane selected from the group consisting essentially of R₃SiO(R′₂SiO)ₐ (R″HSiO)_{b} SiR₃, HR₂SiO(R′₂SiO)_{c} SiR₂H, and, HR₂SiO(R′₂SiO)ₐ (R″HSiO)_{b} SiR₂H, wherein R, R′, and R″, are alkyl groups of 1 to 6 carbon atoms, a is 0 to 250, b is 1 to 250, and c is 0 to 250.

The second component of the siloxane co-polymer is an alpha, omega-diene having the general formula CH₂═CH(CH₂)ₓ CH═CH₂ wherein x has a value of from 1 to 20 which is reacted with the hydridopolysiloxane.

A first additional component is added, said first additional component selected from esters of alkoxylated aromatic alcohol and fatty carboxylic acids to form a siloxane paste.

There is then an additional component which is a second additional component said second additional component being an ester of an alkoxylated aromatic alcohol and a fatty carboxylic acid to form a siloxane polymer base.

In addition, it is contemplated within the scope of this invention to provide a combination of the siloxane polymer base and an additional agent such as, for example, a pharmaceutical, medicament, or the like, and in addition it is contemplated within the scope of this invention to provide a combination of the siloxane polymer base and an additional agent that is selected from the group consisting essentially of skin creams, antiperspirants, deodorants, skin care lotions, moisturizers, acne removers, wrinkle removers, facial cleansers, bath oils, perfumes, colognes, sachets, sunscreens, pre-shave lotions, after shave lotions, liquid soaps, shaving soaps, shaving lathers, shaving gels, hair shampoos, hair conditioners, air sprays, mousses, permanents, depilatories, cuticle coats, make-ups, color, cosmetics, foundations, blushes, lipsticks lip balms, eyeliners, mascaras, oil removers, cosmetic removers, delivery systems for oil and water soluble substances, and powders, including products in the form of sticks, gels, lotions, aerosols, and roll-ons.

Other embodiments of this invention are a method for preparing the combination of the siloxane polymer base and a method for preparing such base and an active used therein.

### DETAILED DESCRIPTION OF THE INVENTION

The —SiH containing polysiloxane is represented by compounds of the formula R₃SiO(R′₂SiO)ₐ (R″HSiO)_{b} SiR₃; HR₂SiO(R′₂SiO)_{c} SiR₂H, and, HR₂SiO(R′₂SiO)ₐ (R″HSiO)_{b} SiR₂H, wherein R, R′, R″, are alkyl groups of 1 to 6 carbon atoms; a is 0 to 250; b is 1 to 250; and c is 0 to 250. Such polysiloxanes are commercially available.

The alpha, omega-diene is a compound of the formula CH₂═CH(CH₂)ₓ CH═CH₂ wherein x has a value of from 1 to 20. Some representative examples of suitable alpha, omega-dienes for use herein are 1,4-pentadiene; 1,5-hexadiene; 1,6-heptadiene; 1,7-octadiene; 1,8-nonadiene; 1,9-decadiene; 1,11-dodecadiene; 1,13-tetradecadiene; and 1,19-eicosadiene.

The addition and crosslinking reaction requires a catalyst to effect the reaction between the SiH containing polysiloxane and the alpha, omega-diene. Suitable catalysts are Group VIII transition metals, i.e., the Nobel metals. Such Noble metal catalysts are described in U.S. Pat. No. 3,923,705, incorporated herein by reference to show platinum catalysts. One preferred platinum catalyst is Karstedt's catalyst, which is described in Karstedt's U.S. Pat. Nos. 3,715,334 and 3,814,730, incorporated herein by reference. Karstedt's catalyst is a platinum divinyl tetramethyl disiloxane complex typically containing about one weight percent of platinum in a solvent such as toluene. Another preferred platinum catalyst is a reaction product of chloroplatinic acid and an organosilicon compound containing terminal aliphatic unsaturation. It is described in U.S. Pat. No. 3,419,593, incorporated herein by reference. The Noble metal catalysts are used in amounts from 0.00001 to 0.5 parts per 110 weight parts of the SiH containing polysiloxane, preferable 0.0001 to 0.02 parts, most preferable is 0.0001 to 0.002 parts.

The inventors, when using “esters of alkoxylated aromatic alcohol and fatty carboxylic acids” mean esters of an alkoxylated aromatic alcohol and a fatty carboxylic acid. The preparation of such materials can be found in U.S. Pat. No. 6,987,195, that issued Jan. 17, 2006 in the name of Pereira, such patent being incorporated herein by reference for what it teaches about such materials and the methods by which they are manufactured.

Carrying out of the process is a matter of combining the SiH containing polysiloxane, the alpha, omega-diene, and an ester of an alkoxylated aromatic alcohol and a fatty carboxylic acid, or a mixture of the esters, and the catalyst, and mixing these ingredients at room temperature until a gel is formed. Higher temperatures to speed up the process can be used, if Desired.

A first additional amount of the ester is then added to the gel, and the resulting mixture is subjected to shear force to form the siloxane paste.

Any type of mixing and shearing equipment may be used to perform these steps such as a batch mixer, planetary mixer, single or multiple screw extruder, dynamic or static mixer, colloid mill, homogenizer, sonolator, or a combination thereof.

The esters can be used in amounts generally within the range of about 20 to 98 percent by weight of the composition, preferably about 50 to 98 percent by weight.

To prepare the siloxane polymer base having a dry to the touch feel, a second addition of an ester, or a combination of esters, is added to the paste described just Supra.

The preferred method for combining the second component into the paste is simple blending rather than shear mixing.

It has been found that the addition of the esters as a first component and the addition of the esters as the final component, provide a dry to the touch material that is especially efficacious for providing topical pharmaceuticals, cosmetics, and the like.

### Example 1

In a 50 ml glass, round-bottomed, glass flask, were placed an organopolysiloxane with the average structure of

(CH₃)₃SiO [(CH₃)₂SiO)]₃₄ (CH₃HSiO)₄ Si (CH₃)₃ (27.84 grams), 1.64 grams of 1,5-hexadiene and 0.0001 grams of platinum #4 catalyst, and mixed. Thereafter, the mixture was diluted using PPG-3 benzyl ether ethylhexanoate in a ratio of 10 weight percent of the mixture to 90 weight % of the ethylhexanoate.

While stirring, with gentle heating, the mixture was allowed to react. Gelation took place within one hour. The gel was transparent in nature.

The gel was then treated with shear force in the presence of PPG-3 benzyl ether ethylhexanoate ( grams) to form a paste.

Thereafter, the paste was treated with (grams) of an additional quantity of PPG-3 benzyl ether ethylhexanoate to form a base. The base has a dry to the touch feel when placed on the skin.

### Example 2

In a 50 ml glass, round-bottomed, glass flask, were placed an organopolysiloxane with the average structure of (CH₃₎₃SiO [(CH₃₎₂SiO)]₅ (CH₃HSiO)₃Si (CH₃)₃, PPG-3 benzyl ether Ethylhexanoate, 1,5-hexadiene and platinum #4 catalyst. The amounts of each are set forth in the table infra. The results are set forth in the table infra.

| | Sample # | | | | |
|---|---|---|---|---|---|
| Material | 2 | 3 | 4 | 5 | 1 |
| Siloxane | 14.24 | 14.24 | 14.24 | 14.24 | 14.24 |
| (gms) | | | | | |
| Ester | 5 | 8.4 | 16.80 | 33.60 | 300.6 |
| (gms) | | | | | |
| Diene | 2.46 | 2.46 | 2.46 | 2.46 | 4.92 |
| (gms) | | | | | |
| Catalyst | 0.00015 | 0.00015 | 0.00015 | 0.0015 | 0.0003 |
| (gms) | | | | | |
| Mix time | 30 | 30 | 30 | 30 | 30 |
| (min.) | | | | | |
| Exotherm | high | med. | slight | little | none |
| Gel time | <2 | 5 | 10 | 50 | >60 |
| (min.) | | | | | |
| Gel | hard | med. | Soft | very soft | liquid |
| (stiffness) | | | | | |
| Add ester | | | | 16.7 | |
| (gms) | | | | (Mushy | |
| | | | | gel) | |

What is claimed is:

1. A composition of matter comprising:
A. a siloxane polymer base comprising a combination of:
a. a siloxane gel, said gel comprised of a first component which is an ester of an alkoxylated aromatic alcohol and a fatty carboxylic acid, or a mixture of such esters, and,
b. a siloxane co-polymer, said siloxane co-polymer comprised of the reaction product of a hydridopolysiloxane selected from the group consisting essentially of:
i. R₃SiO(R′₂SiO)ₐ (R″HSiO)_{b} SiR₃
i. HR₂SiO(R′₂SiO)_{c} SiR₂H, and,
ii. HR₂SiO(R′₂SiO)ₐ (R″HSiO)_{b} SiR₂H,
wherein R, r′, R″, are alkyl groups of 1 to 6 carbon atoms; a is 0 to 250; b is 1 to 250; and c is 0 to 250; with an alpha, omega-diene having the general formula CH₂═CH(CH₂)ₓ CH═CH₂ wherein x has a value of from 1 to 20;
B. a first additional component, said first additional component being an ester of an alkoxylated aromatic alcohol and a fatty carboxylic acid, or a mixture of such esters, to form a siloxane paste;
C. a second additional component, said second additional component selected from the group consisting of an ester of an alkoxylated aromatic alcohol and a fatty carboxylic acid, or a mixture of such esters, to form a siloxane polymer

2. The composition of matter as claimed in claim 1 wherein there is present, in addition, a topical medicament.

3. The composition of matter as claimed in claim 2 wherein the topical medicament is an antibiotic.

4. The composition of matter as claimed in claim 3 wherein the antibiotic is triple antibiotic.

5. The composition of matter as claimed in claim 1 wherein, in addition, there is present an analgesic.

6. The composition of matter as claimed in claim 5 wherein the analgesic is capsaicin.

7. The composition of matter as claimed in claim 5 wherein the analgesic is methyl salicylate.

8. The composition of matter as claimed in claim 5 wherein the analgesic is menthol.

9. The composition of matter as claimed in claim 5 wherein the analgesic is triethanolamine salicylate.

10. The composition of matter as claimed in claim 1 wherein, in addition, there is present at least one essential oil.

11. The composition of matter as claimed in claim 10 wherein the at least one essential oil is a natural mosquito repellent oil.

12. The composition of matter as claimed in claim 10 wherein the at least one essential oil is citronella oil.

13. The composition of matter as claimed in claim 1 wherein, in addition, there is present, a material selected from the group consisting essentially of: antiperspirants, deodorants, skin creams, skin care lotions, moisturizers, acne removers, wrinkle removers, facial cleansers, bath oils, perfumes, colognes, sachets, sunscreens, pre-shave lotions, after shave lotions, liquid soaps, shaving soaps, shaving lathers, shaving gels, hair shampoos, hair conditioners, air sprays, mousses, permanents, depilatories, cuticle coats, make-ups, color, cosmetics, foundations, blushes, lipsticks lip balms, eyeliners, mascaras, oil removers, cosmetic removers, delivery systems for oil and water soluble substances, and powders, including products in the form of sticks, gels, lotions, aerosols, and roll-ons.

14. The composition of matter as claimed in claim 1 wherein, in addition, there is present at least one preservative for the composition.

15. A method of preparing a siloxane polymer base the method comprising:
(A) reacting an Si—H containing polysiloxane of the formula selected from the group consisting of
i. R₃SiO(R′₂SiO)ₐ (R″HSiO)_{b} SiR₃
ii. HR₂SiO(R′₂SiO)_{c} SiR₂H, and,
iii. HR₂SiO(R′₂SiO)ₐ (R″HSiO)_{b} SiR₂H,
wherein R, r′, R″, are alkyl groups of 1 to 6 carbon atoms; a is 0 to 250; b is 1 to 250; and c is 0 to 250; with
(B) an alpha, omega-diene having the general formula CH₂═CH(CH₂)ₓ CH═CH₂ wherein x has a value of from 1 to 20;
(C) conducting the reaction in the presence of a noble metal catalyst and a compound selected from the group consisting of an ester of an alkoxylated aromatic alcohol and a fatty carboxylic acid, or a mixture of such esters, and continuing the reaction until a gel is formed by cross linking and addition of Si—H across double bonds in the alpha, omega-diene;
(D) thereafter, adding a first additional component to the gel and subjecting the first additional component and the gel to shear force until a siloxane paste is formed;
(E) thereafter, adding a second additional component to the paste and blending the paste and second additional component by mixing until a siloxane polymer base is formed.

16. The process of claim 22 wherein, in addition, after or during step (E), there is added at least one active.

17. A product prepared by a process comprising:
(A) reacting an Si—H containing polysiloxane of the formula selected from the group consisting of
i. R₃SiO(R′₂SiO)ₐ (R″HSiO)_{b} SiR₃
ii. HR₂SiO(R′₂SiO)_{c} SiR₂H, and,
iii. HR₂SiO(R′₂SiO)ₐ (R″HSiO)_{b} SiR₂H,
wherein R, r′, R″, are alkyl groups of 1 to 6 carbon atoms; a is 0 to 250; b is 1 to 250; and c is 0 to 250; with
(B) an alpha, omega-diene having the general formula CH₂═CH(CH₂)ₓ CH═CH₂ wherein x has a value of from 1 to 20;
(C) conducting the reaction in the presence of a noble metal catalyst and a compound selected from the group consisting of an ester of an alkoxylated aromatic alcohol and a fatty carboxylic acid, or a mixture of such esters, and continuing the reaction until a gel is formed by cross linking and addition of Si—H across double bonds in the alpha, omega-diene;
(D) thereafter, adding a first additional component to the gel and subjecting the first additional component and the gel to shear force until a siloxane paste is formed;
(E) thereafter, adding a second additional component to the paste and blending the paste and second additional component by mixing until a siloxane polymer base is formed.

18. A product prepared by a process comprising:
(A) reacting an Si—H containing polysiloxane of the formula selected from the group consisting of
i. R₃SiO(R′₂SiO)ₐ (R″HSiO)_{b} SiR₃
ii. HR₂SiO(R′₂SiO)_{c} SiR₂H, and
iii. HR₂SiO(R′₂SiO)ₐ (R″HSiO)_{b} SiR₂H,
wherein R, r′, R″, are alkyl groups of 1 to 6 carbon atoms; a is 0 to 250; b is 1 to 250; and c is 0 to 250; with
(B) an alpha, omega-diene having the general formula CH₂═CH(CH₂)ₓ CH═CH₂ wherein x has a value of from 1 to 20;
(C) conducting the reaction in the presence of a noble metal catalyst and a compound selected from the group consisting of an ester of an alkoxylated aromatic alcohol and a fatty carboxylic acid, or a mixture of such esters, and continuing the reaction until a gel is formed by cross linking and addition of Si—H across double bonds in the alpha, omega-diene.
